(19) United States
(12) Patent Application Publication
(10) Pub. No.: US 2015/0135839 A1
(43) Pub. Date: May 21, 2015

(54) PHOTOACOUSTIC PROBE MODULE AND PHOTOACOUSTIC IMAGING APPARATUS HAVING THE SAME

(71) Applicants: SAMSUNG ELECTRONICS CO., LTD., Suwon-si (KR); SAMSUNG MEDISON CO., LTD., Hongcheon-gun (KR)

(72) Inventors: Jae Kwang LEE, Hwaseong-si (KR); Jung Ho KIM, Seoul (KR); Jung Taek OH, Seoul (KR)

(73) Assignees: SAMSUNG ELECTRONICS CO., LTD., Suwon-si (KR); SAMSUNG MEDISON CO., LTD., Hongcheon-gun (KR)

(21) Appl. No.: 14/550,072

(22) Filed: Nov. 21, 2014

(30) Foreign Application Priority Data
Nov. 21, 2013 (KR) 10-2013-0142525

(51) Int. Cl. G01N 21/17; A61B 5/00

(52) U.S. Cl. CPC G01N 21/1702; A61B 5/0095; G01N 2201/08; G01N 2201/06113; G01N 2021/1708
USPC 73/643

(57) ABSTRACT

A photoacoustic probe module includes an optical system configured to guide a laser beam generated by a laser source such that the laser beam arrives at an object at a target incidence angle and penetrates the object to a target internal depth, and a photoacoustic probe configured to receive acoustic waves emitted from the target depth by the laser beam.

### CROSS-REFERENCE TO RELATED APPLICATION

This application claims priority from Korean Patent Application No. 10-2013-0142525, filed on Nov. 21, 2013 in the Korean Intellectual Property Office, the disclosure of which is incorporated herein by reference in its entirety.

### BACKGROUND

1. Technical Field

Apparatuses and methods consistent with exemplary embodiments relate to a photoacoustic probe module which emits coherent electromagnetic waves toward an object and receives acoustic waves generated from the object, and a photoacoustic imaging apparatus having the same.

2. Description of the Related Art

Medical imaging apparatuses are designed to acquire an image of an object using penetration, absorption, and/or reflection of transmissions that are transmitted and received. The transmissions may include acoustic waves, such as ultrasonic waves, or electromagnetic waves, such as coherent electromagnetic waves and X-rays, from the object. The medical imaging apparatuses are also designed to be used to help diagnose the object using the image. Examples of medical imaging apparatuses include ultrasound imaging apparatuses, photoacoustic imaging apparatuses, and X-ray imaging apparatuses.

Research and development of photoacoustic imaging technologies that may acquire high spatial resolution of ultrasound images and high optical contrast of optical images has actively been conducted.

Photoacoustic imaging technologies may form images of internal structures of an object in a noninvasive manner using photoacoustic effects. Photoacoustic effects are generated by a substance or material which generates acoustic waves by absorbing light or electromagnetic waves.

### SUMMARY

It is an aspect of one or more exemplary embodiments to provide a photoacoustic probe module which may emit coherent electromagnetic waves to be guided by an optical system to arrive at a target internal depth of an object at a target incidence angle, and a photoacoustic imaging apparatus having the same.

According to an aspect of an exemplary embodiment, there is provided a photoacoustic probe module including an optical system configured to guide a laser beam generated by a laser source such that the laser beam arrives at an object at a target incidence angle and penetrates the object to a target internal depth, and a photoacoustic probe configured to receive acoustic waves emitted from the target depth by the laser beam.

The optical system may include a first mirror located along a transmission path of a laser emission direction and is configured to change a direction of the laser beam, and a second mirror configured to reflect the laser beam, having the changed direction, toward the object such that the laser beam arrives at the object at the target incidence angle and penetrates the object to the target internal depth.

The first mirror and the second mirror may be configured to be rotatable.

The optical system may be further configured to guide the laser beam to provide the laser beam with the target incidence angle via rotation of the first mirror and the second mirror.

A distance between the first mirror and the second mirror may be configured to be adjustable.

The optical system may be further configured to guide the laser beam such that the laser beam arrives at the target depth via adjustment of the distance between the first mirror and the second mirror.

The optical system may include a prism configured to guide the laser beam such that the emitted laser beam has the target incidence angle.

The optical system may be coupled to the photoacoustic probe and is configured to move in a longitudinal direction along the photoacoustic probe.

The optical system may be further configured to move along the photoacoustic probe and guide the laser beam such that the laser beam penetrates to the target depth.

The module may further include an optical fiber configured to transmit the laser beam generated by the laser source to the optical system.

According to an aspect of another exemplary embodiment, there is provided a photoacoustic imaging apparatus including a laser source configured to generate a laser beam, a photoacoustic probe module including an optical system configured to guide the laser beam such that the laser beam arrives at an object at a target incidence angle and penetrates the object to a target internal depth, and a photoacoustic probe configured to receive acoustic waves emitted from the target depth by the laser beam, and an image processor configured to produce a photoacoustic image based on the received acoustic waves.

The optical system may include a first mirror located along a transmission path of a laser emission direction and is configured to change a direction of the laser beam, and a second mirror configured to reflect the laser beam, having the changed direction, toward the object such that the laser beam arrives at the object at the target incidence angle and penetrates the object to the target internal depth.

The first mirror and the second mirror may be configured to be rotatable.

The optical system may be further configured to guide the laser beam to provide the laser beam with the target incidence angle via rotation of the first mirror and the second mirror.

A distance between the first mirror and the second mirror may be configured to be adjustable.

The optical system may be further configured to guide the laser beam such that the laser beam arrives at the target depth via adjustment of the distance between the first mirror and the second mirror.

The optical system may include a prism configured to guide the laser beam such that the emitted laser beam has the target incidence angle.

The optical system may be coupled to the photoacoustic probe and is configured to move in a longitudinal direction along the photoacoustic probe.

The optical system may be further configured to move along the photoacoustic probe and guide the laser beam such that the laser beam penetrates to the target depth.

The photoacoustic probe module may further include an optical fiber configured to transmit the laser beam generated by the laser source to the optical system.

### BRIEF DESCRIPTION OF THE DRAWINGS

The above and/or other aspects will become apparent and more readily appreciated from the following description of exemplary embodiments, taken in conjunction with the accompanying drawings, in which:

FIG. 1 is a view schematically showing a configuration of a photoacoustic probe module in accordance with an exemplary embodiment;

FIGS. 2A, 2B, and 2C are views showing a coupling configuration between an optical system and a photoacoustic probe according to an exemplary embodiment;

FIGS. 3A, 3B, 3C, and 3D are views showing a coupling configuration between an optical system and a photoacoustic probe according to an exemplary embodiment;

FIG. 4 is a view showing laser emission toward a photoacoustic probe module according to an exemplary embodiment;

FIG. 5 is a view showing an optical system applied to a photoacoustic probe module, similar to that shown in of FIG. 1, according to an exemplary embodiment;

FIGS. 6A, 6B, 6C, and 6D are views showing guidance stages of a laser beam through an optical system and a photoacoustic probe, similar to that shown in FIG. 5, according to an exemplary embodiment;

FIGS. 7A, 7B, and 7C are views showing control of a laser beam incidence angle by an optical system in accordance with an exemplary embodiment;

FIGS. 8A, 8B, and 8C are views showing control of an internal depth of an object, at which a laser beam arrives, by an optical system in accordance with an exemplary embodiment;

FIGS. 9 and 10 are views showing an optical system applied to a photoacoustic probe module, similar to that shown in of FIG. 1, according to an exemplary embodiment;

FIGS. 11A, 11B, and 11C are views showing control of an internal depth of an object, at which a laser beam arrives, by the optical system in accordance with another exemplary embodiment;

FIG. 12 is a block diagram showing a photoacoustic imaging apparatus including a photoacoustic probe module in accordance with an exemplary embodiment;

FIG. 13 is a flowchart showing a method of guiding a laser to an object in accordance with an exemplary embodiment;

FIG. 14 is a flowchart showing a method of setting an optical system in accordance with an exemplary embodiment; and

FIG. 15 is a flowchart showing a method of setting an optical system in accordance with an exemplary embodiment.

### DETAILED DESCRIPTION

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The progression of processing operations described is an example; however, the sequence of and/or operations is not limited to that set forth herein and may be changed as is known in the art, with the exception of operations necessarily occurring in a particular order. In addition, respective descriptions of well-known functions and constructions may be omitted for increased clarity and conciseness.

Additionally, exemplary embodiments will now be described more fully hereinafter with reference to the accompanying drawings. The exemplary embodiments may, however, be embodied in many different forms and should not be construed as being limited to the embodiments set forth herein. These embodiments are provided so that this disclosure will be thorough and complete and will fully convey the exemplary embodiments to those of ordinary skill in the art. The scope is defined not by the detailed description but by the appended claims. Like numerals denote like elements throughout.

Reference will now be made in detail to a photoacoustic probe module and a photoacoustic imaging apparatus having the same in accordance with the exemplary embodiments, examples of which are illustrated in the accompanying drawings, wherein like reference numerals refer to like elements throughout.

Photoacoustic Imaging (PAI) may be suitable for imaging tissue using a combination of high spatial resolution of ultrasound images and high optical contrast of optical images. When coherent electromagnetic waves, which may be called laser beams, having wavelengths of nanometers are emitted toward tissues, the tissues absorb short electromagnetic pulses of coherent electromagnetic waves, causing instantaneous generation of acoustic pressure by thermo-elastic expansion in tissue regions that serve as an initial ultrasonic wave source. The resulting ultrasonic waves arrive at a surface of tissues with different delays, allowing for the formation of a photoacoustic image.

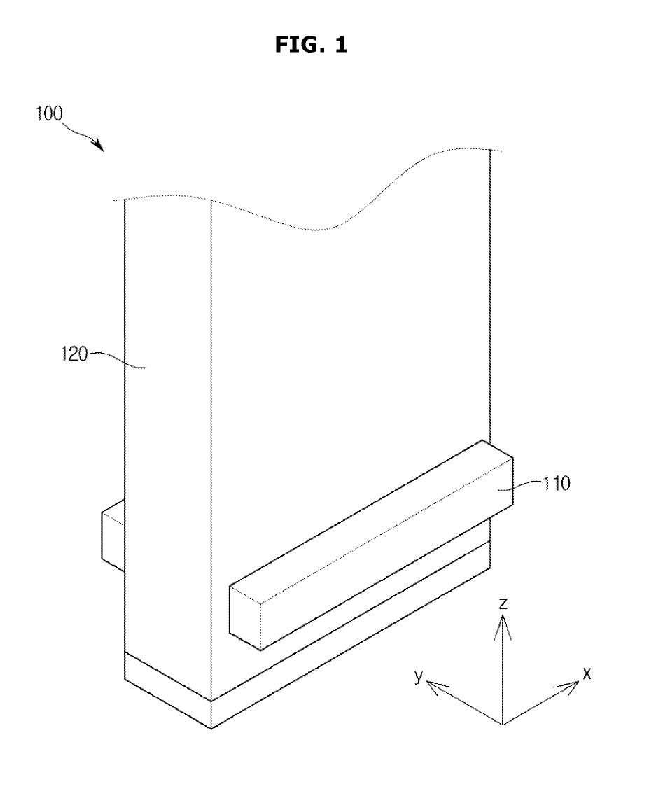

FIG. 1 is a view schematically showing a configuration of a photoacoustic probe module in accordance with an exemplary embodiment.

Referring to FIG. 1, a photoacoustic probe module 100 in accordance with an exemplary embodiment may include an optical system 110 configured to guide a laser beam generated by a laser source such that the laser beam arrives at a target internal depth of an object/tissue at a target incidence angle, and a photoacoustic probe 120 that is configured to then receive acoustic waves emitted from the target depth at which the laser beam has arrived.

The optical system 110 may guide a laser beam to the object. Specifically, to guide a laser beam to the object, the optical system 110 may control an angle between the laser and a surface of the object (hereinafter referred to as incidence angle) and a point, at which the laser arrives, present on the extension of a center axis of the photoacoustic probe 120 (hereinafter referred to as an internal depth of the object).

Variation in the incidence angle of the laser beam causes variation in the laser absorption rate of the surface of the object, e.g., the skin. As experimentally provided, when the object is a human body, maximum absorption rate is derived when the laser is emitted into the object at an incidence angle of approximately 55 degrees. Accordingly, the optical system 110 may guide the laser to the object at a target incidence angle to ensure efficient absorption.

In addition, because a photoacoustic image may be produced by receiving acoustic waves from a laser beam arrival region, the optical system 110 may guide the laser such that the laser arrives at a target depth for imaging.

Referring again to FIG. 1, the photoacoustic probe 120 may receive acoustic waves generated from within the object to be imaged when the laser beam is guided to the object through the optical system 110.

The photoacoustic probe 120 may include a transducer to convert the received acoustic waves into an electrical signal. The transducer may include a piezoelectric layer to convert an acoustic signal into an electrical signal, a matching layer disposed on a front surface of the piezoelectric layer, and a backing layer disposed on a rear surface of the piezoelectric layer.

Piezoelectric effects refer to generation of voltage when mechanical pressure is applied to a material, and materials exhibiting these effects are referred to as piezoelectric materials. That is, a piezoelectric material converts mechanical vibration energy into electrical energy.

The piezoelectric layer is formed of a piezoelectric material and converts a received acoustic signal into an electrical signal.

The piezoelectric material of the piezoelectric layer may include lead zirconate titanate (PZT) ceramics, PZMT single-crystals made of magnesium noibate and lead zirconate titanate solid solution, PZNT single-crystals made of zincniobdate, and lead zirconate titanate solid solution, etc.

The matching layer, disposed on the front surface of the piezoelectric layer, reduces a difference of acoustic impedances between the piezoelectric layer and the object to ensure effective transmission of acoustic waves from the object to the piezoelectric layer. The matching layer may have a single layer or multilayer form, and both the matching layer and the piezoelectric layer may be divided into a plurality of units having a predetermined width by dicing.

The backing layer, disposed on the rear surface of the piezoelectric layer, absorbs acoustic waves generated in the piezoelectric layer and prevents radiation of acoustic waves from the rear surface of the piezoelectric layer, thereby serving to prevent image distortion. The backing layer may have a multilayer form to enhance attenuation or interception of ultrasonic waves.

The photoacoustic probe 120 which is configured to receive acoustic waves may be an ultrasound probe configured to receive ultrasonic waves where acoustic waves are understood to be ultrasonic waves in the field of photoacoustic imaging. Thus, conventional ultrasound probes used in ultrasonic diagnosis may also be used in photoacoustic diagnosis.

Referring again to FIG. 1, the optical system 110 and the photoacoustic probe 120 may be coupled to each other to form a single unit. For example, the optical system 110 and the photoacoustic probe 120 may be integrated. That is, the optical system 110 and the photoacoustic probe 120 may be mounted in a housing. Alternatively, the optical system 110 may be separably coupled to the photoacoustic probe 120.

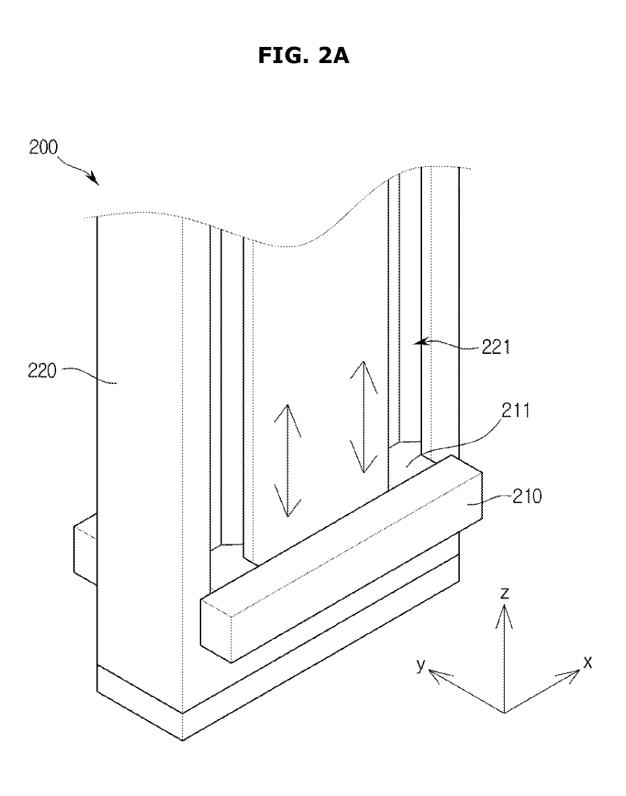

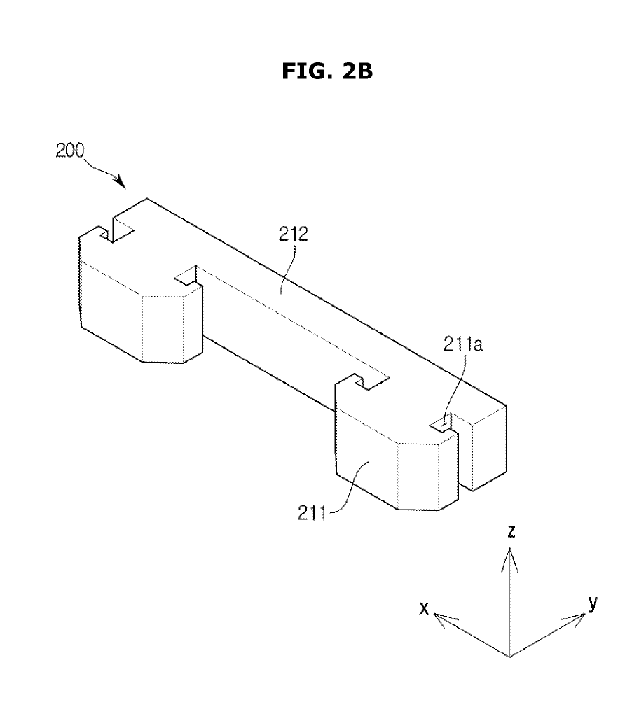

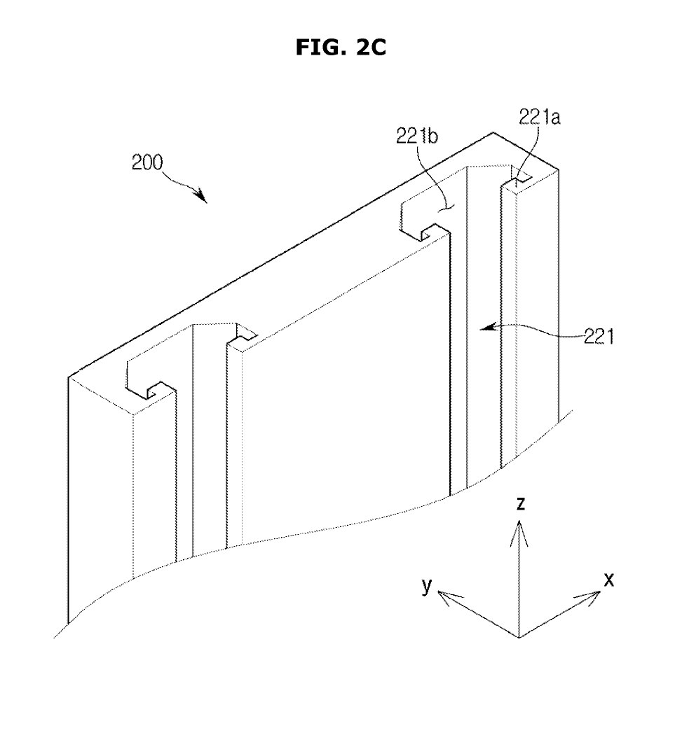

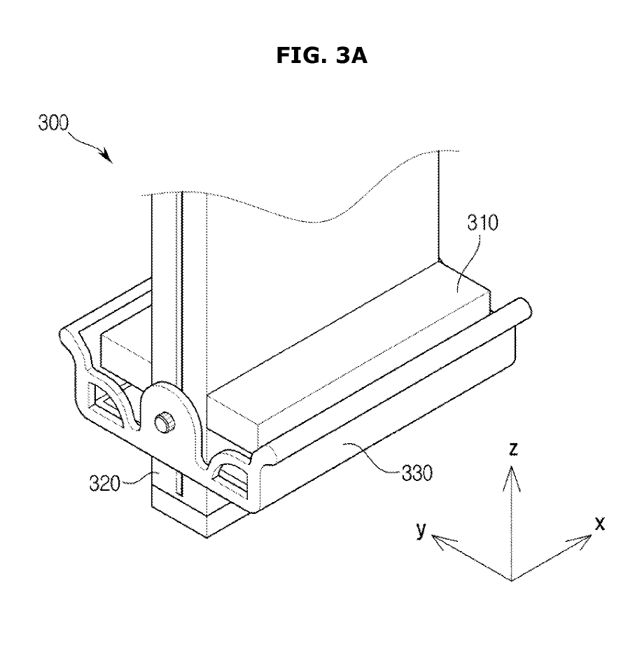

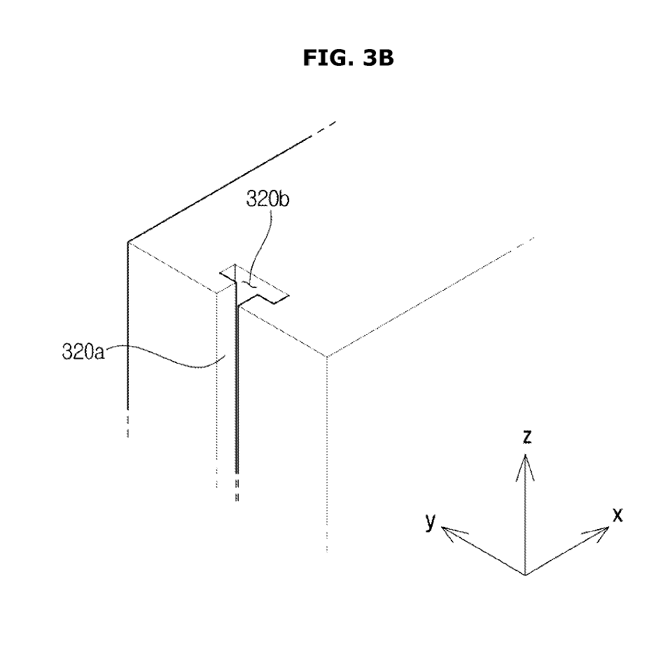

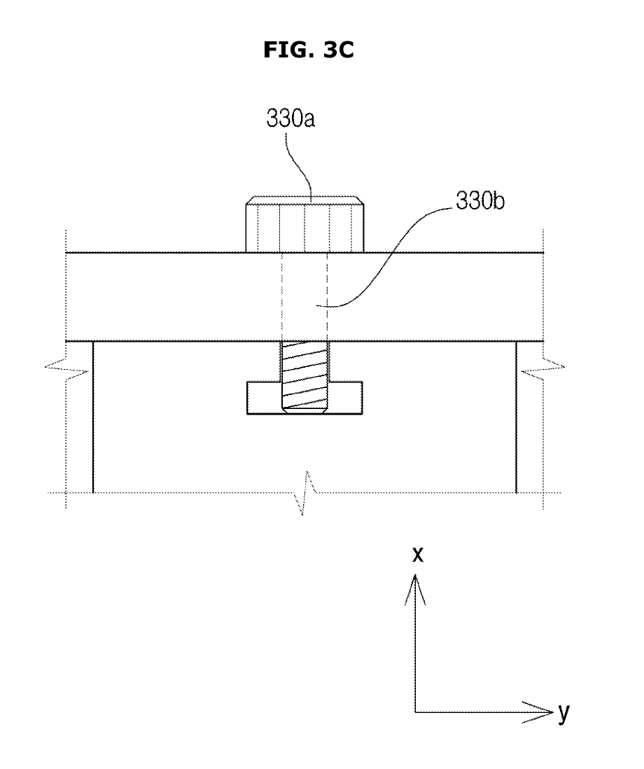

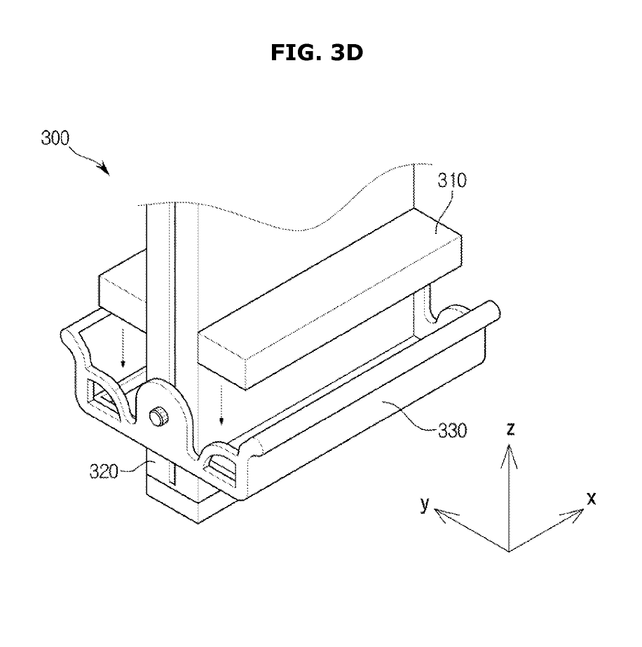

Hereinafter, coupling configurations between an optical system and a photoacoustic probe will be described with reference to FIGS. 2A to 2C and FIGS. 3A to 3D.

FIGS. 2A to 2C are views showing an exemplary embodiment of a coupling configuration of a photoacoustic probe module 200 between a photoacoustic probe 220 and an optical system 210.

As shown in FIG. 2A, the optical system 210 may be directly coupled to the photoacoustic probe 220. In particular, the optical system 210 may be slidably coupled to the photoacoustic probe 220 and thus, is movable along the photoacoustic probe 220.

The optical system 210, as shown in FIG. 2B, may include a moving member 211 coupled to a rail 221, as shown in FIGS. 2A and 2C, on the photoacoustic probe 220 to slide along the rail 221, and an optical member 212 coming into surface contact with and connected to the moving member 211, where the optical member 212 serves to guide a laser beam.

The moving member 211 may take the form of a protruding member as exemplarily shown in FIG. 2B and conversely, may take the form of a recessed member. The form of the moving member 211 is not limited to the embodiment of FIG. 2B so long as it may be coupled to a rail 221 of the photoacoustic probe 220 that will be described hereinafter.

The moving member 211 may have an indented portion 211a for stable coupling with the photoacoustic probe 220. As the indented portion 211a of the moving member 211 may be snap-fitted with a raised portion 221a formed at the rail 221 that will be described hereinafter, the optical system 210 may be fixed in the X and Y-axis while maintaining the ability to move longitudinally along the Z-axis.

FIG. 2C is a view showing the rail 221 formed at the exterior of the housing of the photoacoustic probe. The rail 221, which may also be called a support member, may be installed in a longitudinal direction along the photoacoustic probe 220, and the moving member 211 may be coupled to the rail 221.

As mentioned above, the rail 221 may have a form corresponding to the form of the moving member 211. To enable coupling of the moving member 211 in the form of a protruding member as exemplarily shown in FIG. 2B, the rail 221 may take the form of a groove as exemplarily shown in FIG. 2C. Conversely, according to another exemplary embodiment, the rail 221 may take the form of a protrusion when the moving member 211 takes the form of a recessed member.

The rail 221 may include a raised portion 221a to be snap-fitted into the indented portion 211a of the moving member 211 to fix the optical system 210 in the X and Y-axis. When the optical system 210 is separable from the photoacoustic probe 220, as exemplarily shown in FIG. 2C, the rail 221 may further include a coupling region 221b at one end thereof, through which the moving member 210 is coupled to the rail 221. Specifically, the moving member 211 of the optical system 210 may be coupled to the rail 221 through the coupling region 221b, or the optical system 210 may be separated from the rail 221 through the coupling region 221b.

The optical system 210, coupled to the photoacoustic probe 220 as described above, may slide along the rail 221 up and down in a longitudinal direction along the Z-axis. When the rail 221 is installed along a longitudinal direction of the photoacoustic probe 220 as exemplarily shown in FIG. 2A, the optical system 210 coupled to the photoacoustic probe 220 may move along the longitudinal direction (as designated by arrows) of the photoacoustic probe 220.

FIGS. 3A to 3D are views showing another exemplary embodiment of a coupling configuration of a photoacoustic probe module 300 between an optical system and a photoacoustic probe of the photoacoustic probe module 300.

As shown in FIG. 3A, an optical system 310 may be coupled to a photoacoustic probe 320 by a bracket 330 that is configured to hold the optical system 210 while being connected to the photoacoustic probe 320. In this case, the bracket 330 may be coupled to the photoacoustic probe 320, and the optical system 310 may be received in the bracket 330. When the bracket 330 is slidably coupled to the photoacoustic probe 320, the optical system 310 received in the bracket 330 is also slidable.

Referring to FIGS. 3B and 3C, the bracket 330 may have a through-hole 330b perforated in a given direction, and a fixing member 330a may be fastened in the through-hole 330b.

The through-hole 330b may be perforated in the bracket 330 in the X-axis, and a spiral groove may be formed at the inner circumference of the through-hole 330b.

The fixing member 330a may be fastened in the through-hole 330b having the above described form. To this end, the fixing member 330a may have a spiral ridge formed at the outer circumference thereof so as to be engaged with the spiral groove of the through-hole 330b. After the fixing member 330a is positioned at the entrance of the through-hole 330b, the fixing member 330a is repeatedly rotated in a given direction, thereby being inserted into the through-hole 330b until completely fastened in the through-hole 330b.

The fixing member 330a, fastened in the through-hole 330b, may be fitted into a rail 320a formed in the photoacoustic probe 320. Consequently, the bracket 330 may move along the rail 320a.

When the bracket 330 is separable from the photoacoustic probe 320, as exemplarily shown in FIG. 3B, the rail 320a may include a coupling region 320b at one end thereof, through which the fixing member 330a is coupled. Specifically, the fixing member 330a of the bracket 330 may be coupled to the rail 320a through the coupling region 320b, or the bracket 330 may be separated from the rail 320a through the coupling region 320b.

A position of the bracket 330 may be fixed by the fixing member 330a. Referring to FIG. 3C, the fixing member 330a may be inserted into the through-hole 330b and also be fitted into the rail 320a. In this case, the fixing member 330a may be repeatedly rotated until the bottom of the fixing member 330a comes into contact with the rail 320a. When the fixing member 330a tightly comes into contact with the rail 320a, friction is generated between the end tip of the fixing member 330a and the back internal wall of the rail 320a during movement of the bracket 330 in the Z-axis, causing the bracket 330 to be fixed at a specific position of the photoacoustic probe 320.

Once a position of the bracket 330 has been fixed, as shown in FIG. 3D, the optical system 310 may be received in the bracket 330. When the bracket 330 is fixed at a selected position and the optical system 310 is received in the bracket 330, the effects of fixing the optical system 310 at the selected position may be accomplished.

It will be appreciated that FIGS. 2A to 2C and FIGS. 3A to 3D illustrate exemplary embodiments of coupling between the optical system 110; 210, or 310 and the photoacoustic probe 120; 220, or 320 by way of example, and any other coupling configurations between the optical system and the photoacoustic probe may be applied.

For convenience of description, the following description assumes that the optical system 110 is directly coupled to the photoacoustic probe 120 in a sliding manner.

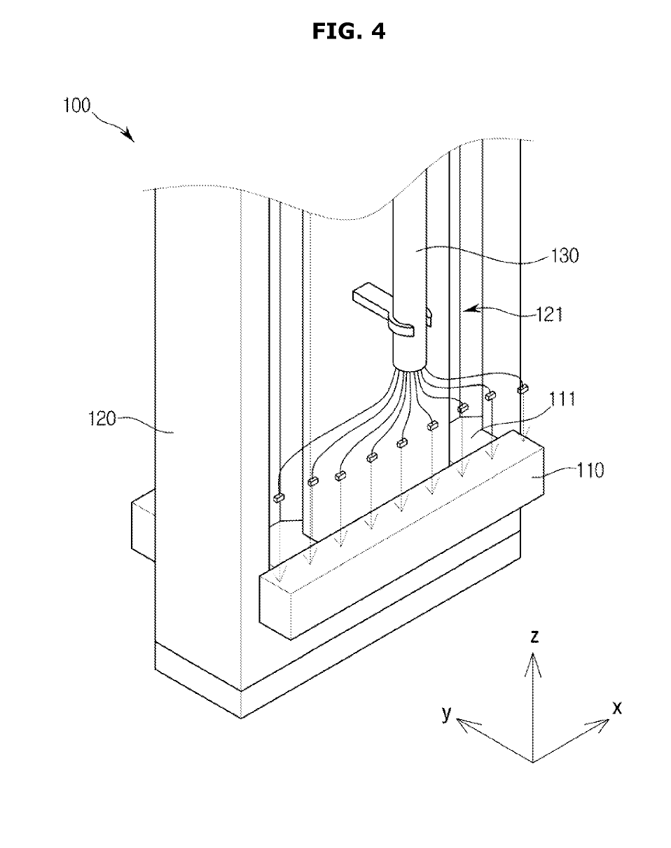

FIG. 4 is a view showing an exemplary embodiment of laser beam emission toward an optical system of a photoacoustic probe module.

The photoacoustic probe module 100 may further include an optical fiber 130 configured to transmit coherent electromagnetic waves, which may also be called a laser beam, generated by a laser source toward the optical system 110. The photoacoustic probe module 100 may include one or more optical fibers 130. Upon provision of the plural optical fibers 130, as exemplarily shown in FIG. 4, the plural optical fibers 130 may be a bundle of optical fibers. Thus, for brevity, the bundle of optical fibers is referred to as the optical fiber 130.

The optical fiber 130 and the photoacoustic probe 120 may be integrally fixed to each other. To this end, as exemplarily shown in FIG. 4, a support member 121 may be used to fix the optical fiber 130 and the photoacoustic probe 120 to each other. In this case, the support member 121 is movable in a longitudinal direction of the photoacoustic probe 120, and thus the optical fiber 130 is movable in a longitudinal direction of the photoacoustic probe 120.

Alternatively, the photoacoustic probe 120 and the optical fiber 130 may be mounted in a housing. Note that the photoacoustic probe module 100 is not limited to illustrations of the above embodiments and has no limit with regard to a connection relationship with the optical fiber 130 and the photoacoustic probe 120.

The optical fiber 130 transmits a laser beam generated by a laser source 160 toward the optical system 110, and the optical system 110 guides the transmitted laser to the object. Hereinafter, various embodiments of the optical system 110 will be described for explanation of guidance of a laser beam by the optical system 110.

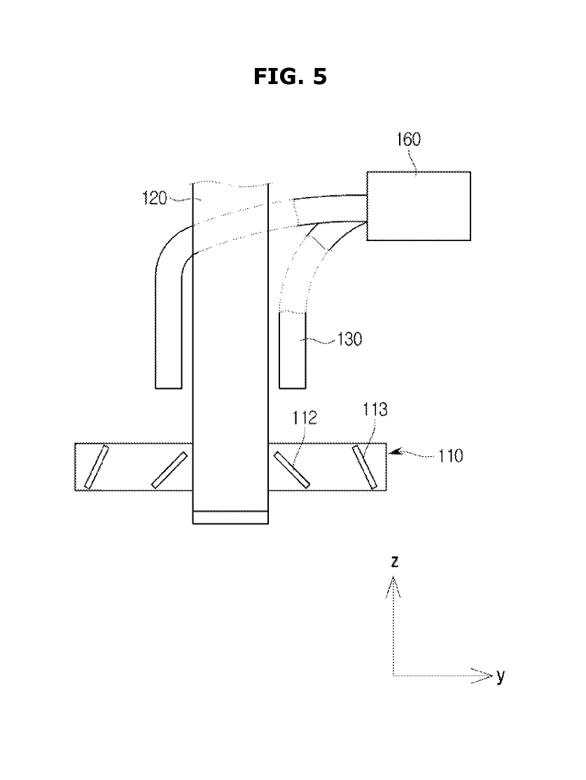

FIG. 5 is a view showing an exemplary embodiment of the optical system applied to the photoacoustic probe module of the above described embodiment.

Referring to FIG. 5, the optical system 110 in accordance with one embodiment may include a first mirror 112 positioned along the transmission path of a laser emission direction to change a direction of the laser beam, and a second mirror 113 configured to reflect the laser beam, having the changed direction, toward the object such that the laser beam arrives at a target internal depth of the object at a target incidence angle.

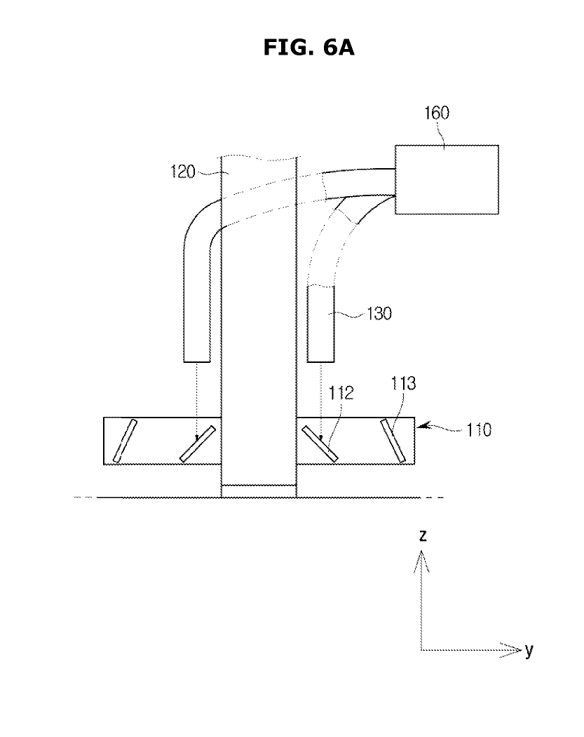

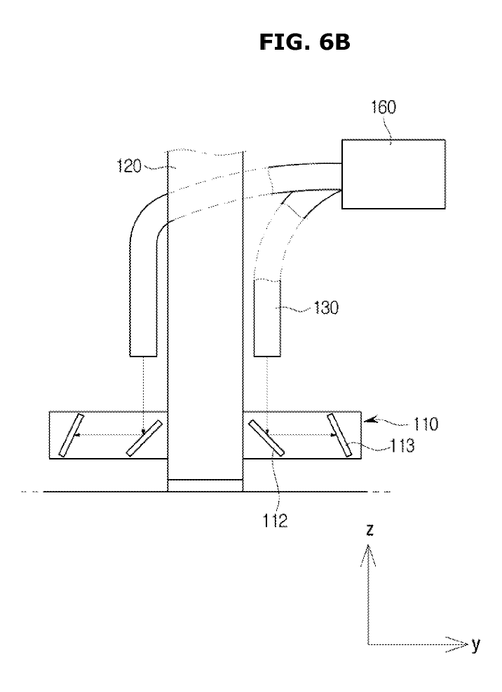

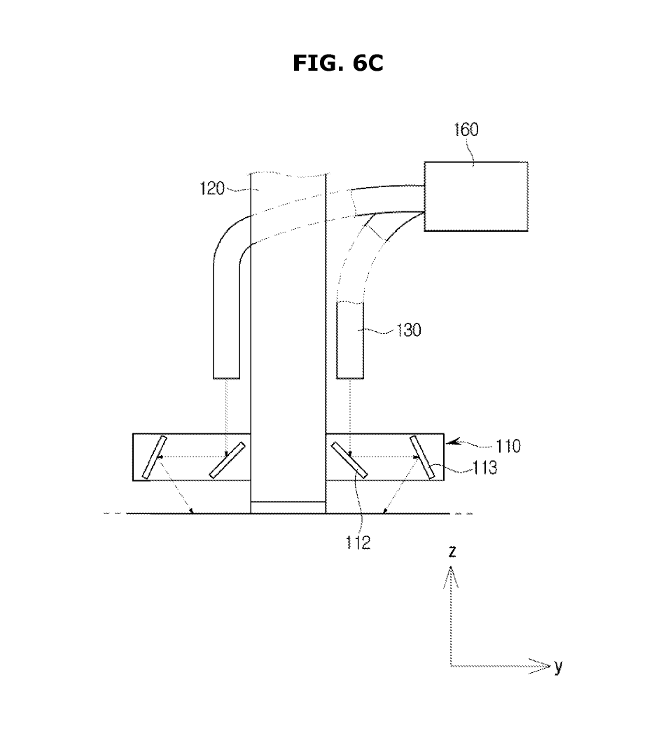

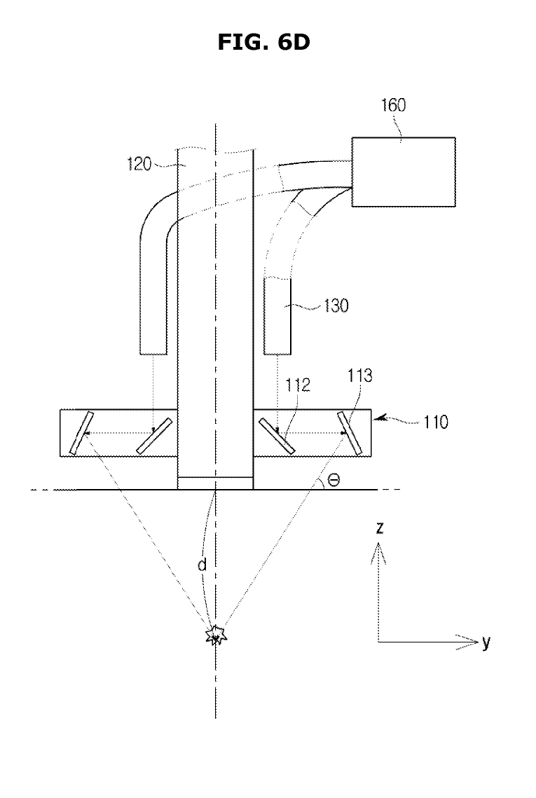

FIGS. 6A to 6D are views explaining guidance of the laser using the optical system and the photoacoustic probe as shown in FIG. 5.

As exemplarily shown in FIG. 6A, the optical fiber 130 may transmit a laser beam generated by the laser source 160 toward the optical system 110. Specifically, one end of the optical fiber 130 may be connected to the laser source 160 to receive the laser beam. The other end of the optical fiber 130 may emit the received laser beam outward. For convenience of description, herein, the laser beam is emitted along the Z-axis.

Referring to FIG. 6B, the laser beam emitted from the optical fiber 130 may be reflected by the first mirror 112 of the optical system 110. In this case, the first mirror 112 may be positioned along the path of a laser beam emission direction from the optical fiber 130. The laser beam emitted in the Z-axis is reflected by the first mirror 112, thereby changing direction toward the second mirror 113.

As exemplarily shown in FIG. 6C, the laser beam, a direction of which has been changed by the first mirror 112, may be again changed in direction by the second mirror 113. The laser beam reflected by the second mirror 113 is reflected toward the object, and thus the second mirror 113 may serve to guide the laser beam to the object.

FIG. 6D is a view showing a case in which the laser beam is finally guided toward a particular point at a particular depth while penetrating at a particular angle toward the object by the optical system.

Particularly, the laser beam emitted from the optical fiber 130 is reflected by the first mirror 112, and in turn the laser beam reflected by the first mirror 112 is again reflected by the second mirror 113. The laser beam reflected by the second mirror 113 is emitted toward the object at an incidence angle 8 with the surface of the object. Then, the laser beam, having passed through the surface of the object at the incidence angle 8, finally arrives at a point located at an internal depth d of the object.

The first mirror 112 and the second mirror 113 of the optical system 110 may be rotatable. The laser incidence angle relative to the object may be controlled via rotation of the first mirror 112 and the second mirror 113. As described above with reference to FIG. 3, the absorption rate of the laser beam at the surface of the object (the skin of the human body) varies based on the laser incidence angle, and thus the laser incidence angle may be optically controlled using the first mirror 112 and the second mirror 113.

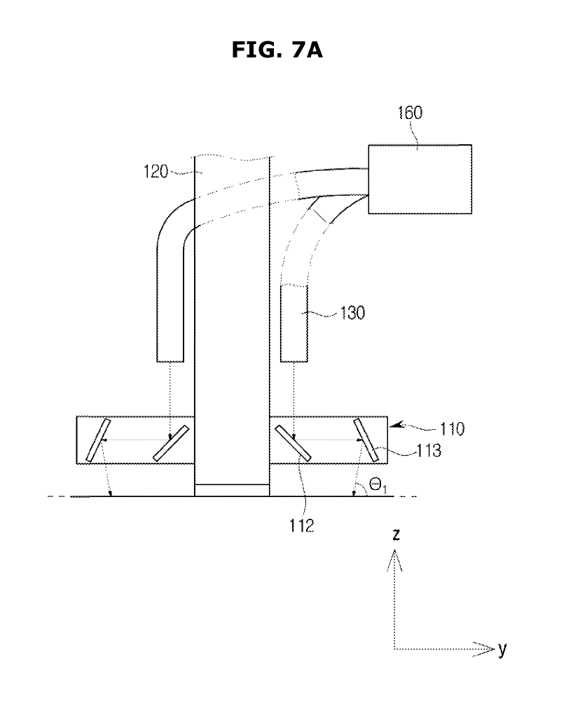

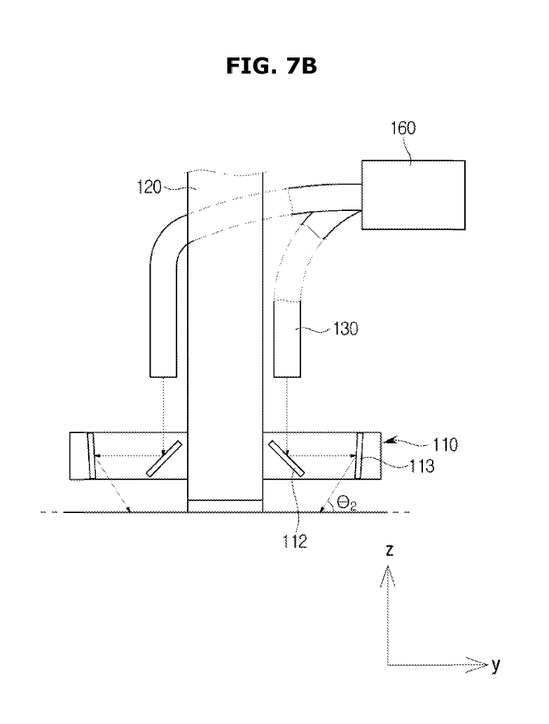

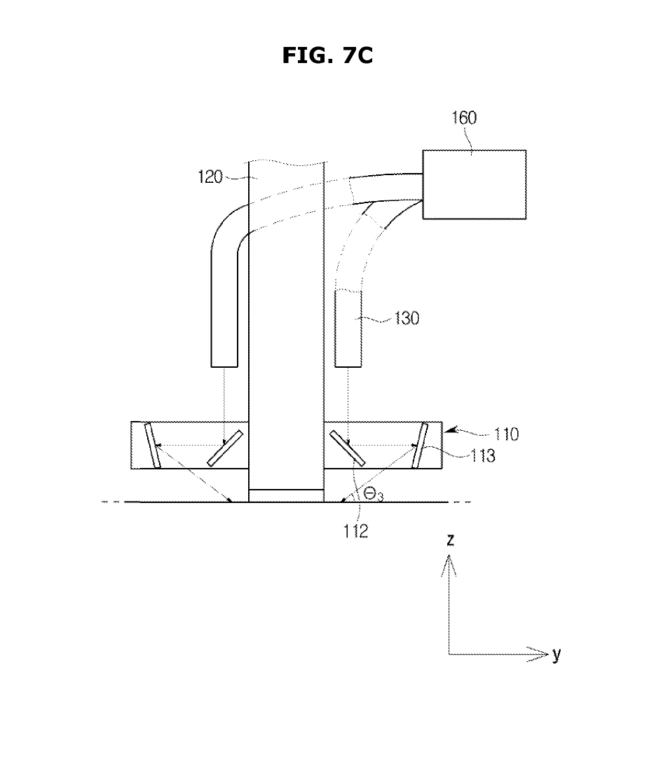

FIGS. 7A, 7B, and 7C are views showing control of the laser incidence angle by the optical system in accordance with an exemplary embodiment.

To vary the laser incidence angle relative to the object, the first mirror 112 may be kept fixed and the second mirror 113 may be rotated. FIGS. 7A to 7C show variation in the laser incidence angle via rotation of the second mirror 113.

For example, the second mirror 113 may be rotated such that a reflecting face thereof becomes substantially perpendicular to the surface of the object as exemplarily shown in FIGS. 7A to 7C. As a result, it will be appreciated that the laser incidence angle is gradually reduced in the order of θ₁, θ₂, and θ₃.

Although FIGS. 7A to 7C illustrate the case of controlling the incidence angle by keeping the first mirror 112 fixed and rotating the second mirror 113, the incidence angle may be controlled by keeping the second mirror 113 fixed and rotating the first mirror 112, or may be controlled by rotating both the first mirror 112 and the second mirror 113.

Through rotation of the first mirror 112 or the second mirror 113 as described above, laser emission may be controlled such that the laser beam has an optimum incidence angle to acquire a photoacoustic image. In this case, the optimum incidence angle means an incidence angle to ensure maximum laser beam absorption rate at the surface of the object (the skin of the human body).

To set the optical system 110 to a target laser incidence angle, a user may directly rotate the first mirror 112 or the second mirror 113, or the first mirror 112 or the second mirror 113 may be rotated based on internal operation of an apparatus.

In addition, a distance between the first mirror 112 and the second mirror 113 may be adjusted to control an internal depth of the object at which the laser arrives. Controlling the laser beam to arrive at a selected internal depth of the object ensures increased laser beam energy at a region corresponding to the depth. It may be important to control laser beam emission to a selected depth because emission of greater laser beam energy enables acquisition of more accurate information. To this end, adjustment of the distance between the first mirror 112 and the second mirror 113 may be implemented.

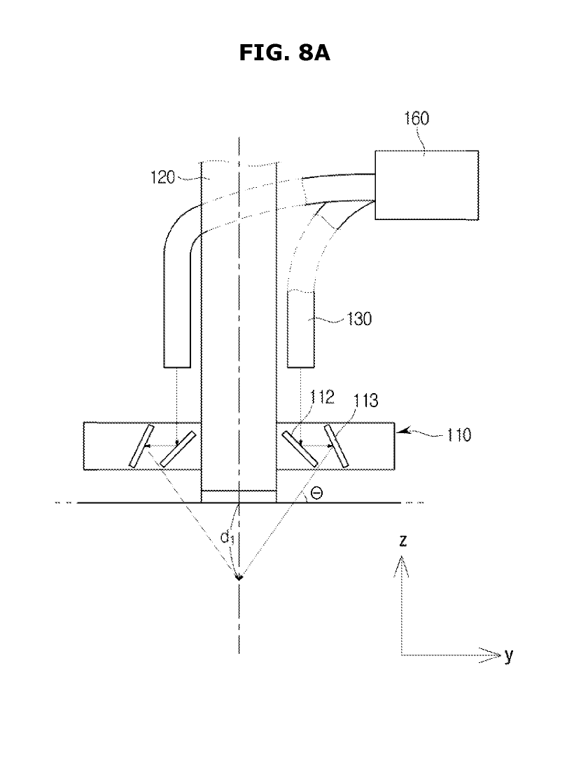

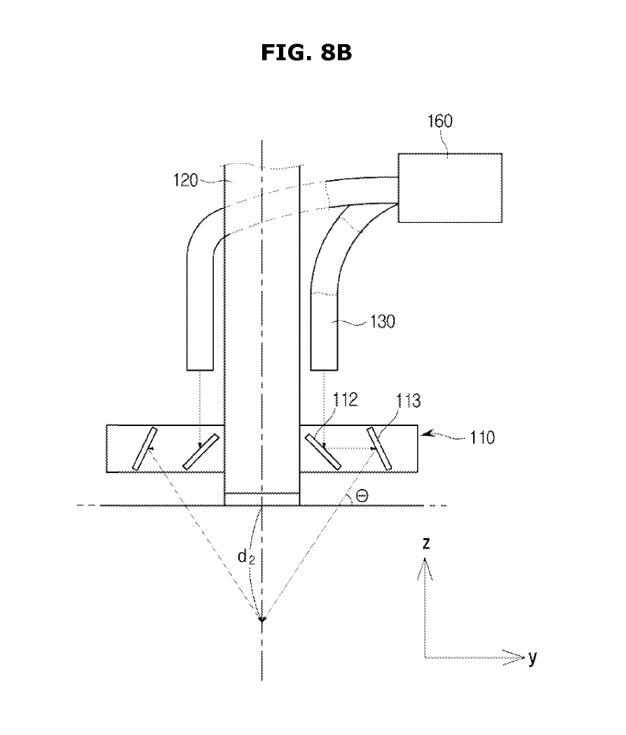

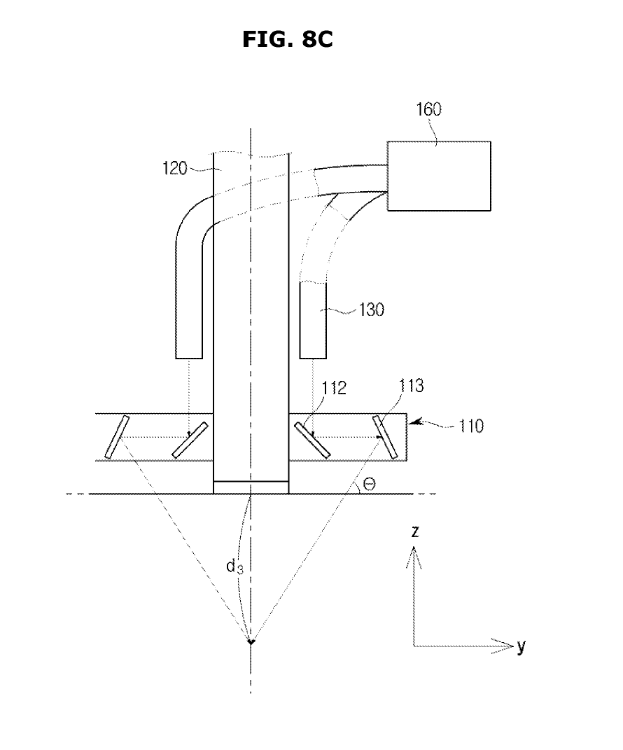

FIGS. 8A, 8B, and 8C are views showing control of an internal depth of the object, at which the laser beam arrives, by the optical system in accordance with one embodiment.

To vary an internal depth of the object, at which the laser beam arrives, the first mirror 112 may be kept fixed and a position of the second mirror 113 may be shifted. FIGS. 8A to 8C illustrate the case of controlling the internal depth of the object, at which the laser arrives, via movement of the second mirror 113.

The second mirror 113 is moved away from the first mirror 112 with increasing distance from FIG. 8A to FIG. 8C. As a result, it will be appreciated that the internal depth of the object, at which the laser beam arrives, gradually increases in the order of d₁, d₂, and d₃.

Although FIGS. 8A to 8C illustrate the case of controlling the internal depth of the object at which the laser beam arrives by keeping the first mirror 112 fixed and moving the second mirror 113, the arrival depth may be controlled by keeping the second mirror 113 fixed and moving the first mirror 112, or may be controlled by moving both the first mirror 112 and the second mirror 113.

Through movement of the first mirror 112 or the second mirror 113 as described above, the laser beam may be emitted toward a target region for acquisition of a photoacoustic image. In this case, a depth of the target region for acquisition of a photoacoustic image from the surface of the object is referred to as a target depth.

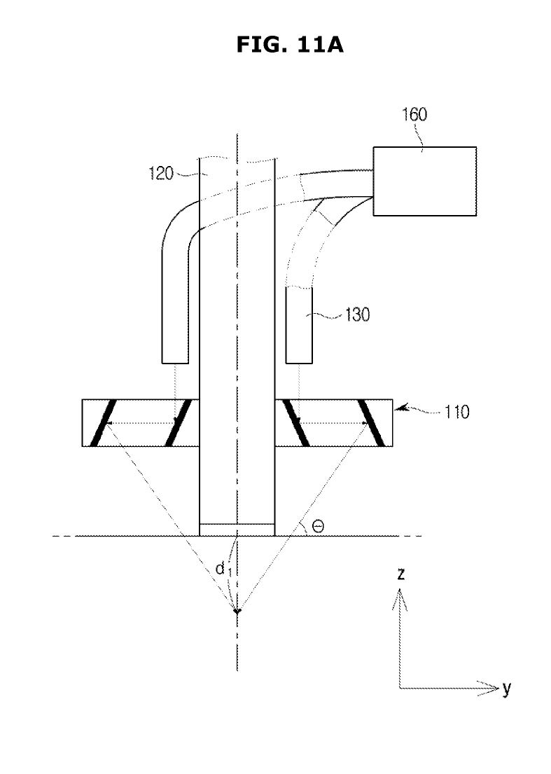

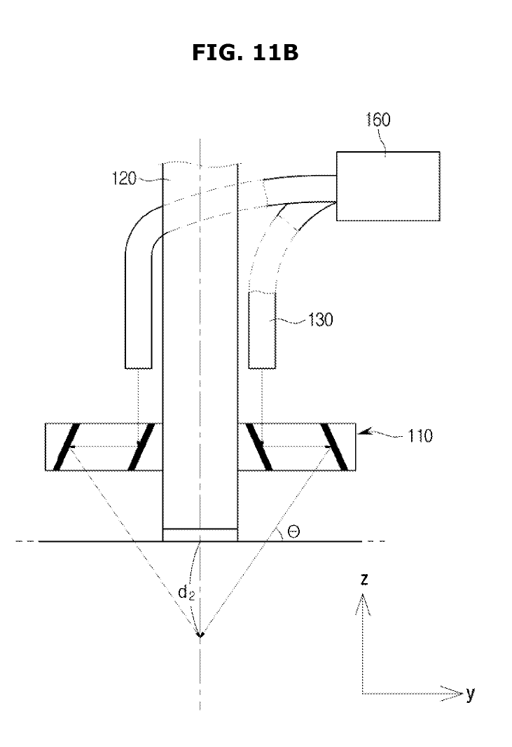

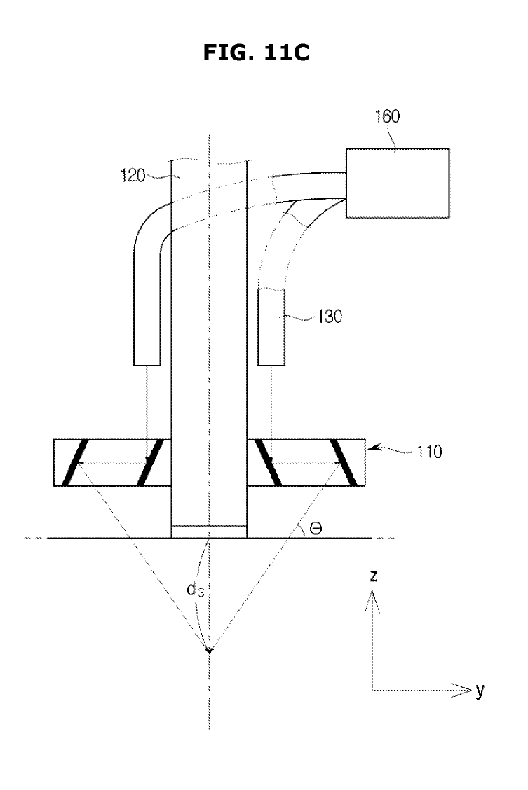

According to another exemplary embodiment, the first mirror 112 and the second mirror 113 may be moved longitudinally along the photoacoustic probe 120 to control arrival of the laser at a target depth. This control method is similar to that using a prism that will be described hereinafter, and will be described below with reference to FIGS. 11A to 11C.

To set the optical system 110 to guide the laser beam to a target depth, the user may directly move the first mirror 112 or the second mirror 113, or the first mirror 112 or the second mirror 113 may be moved based on internal operation of an apparatus.

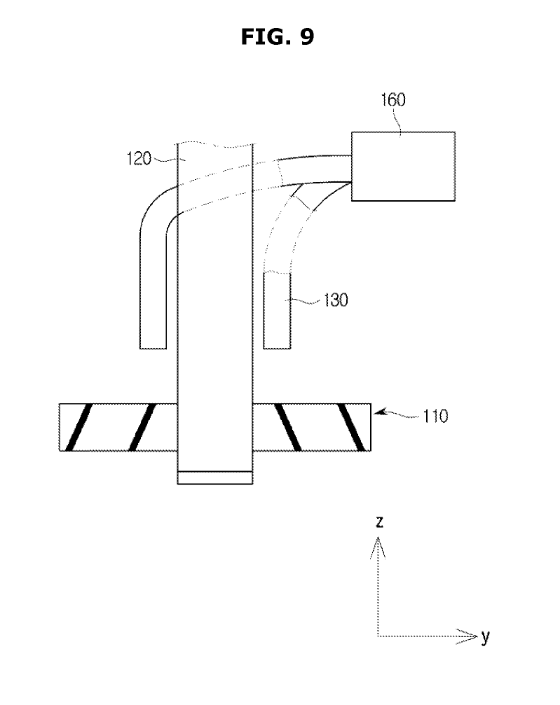

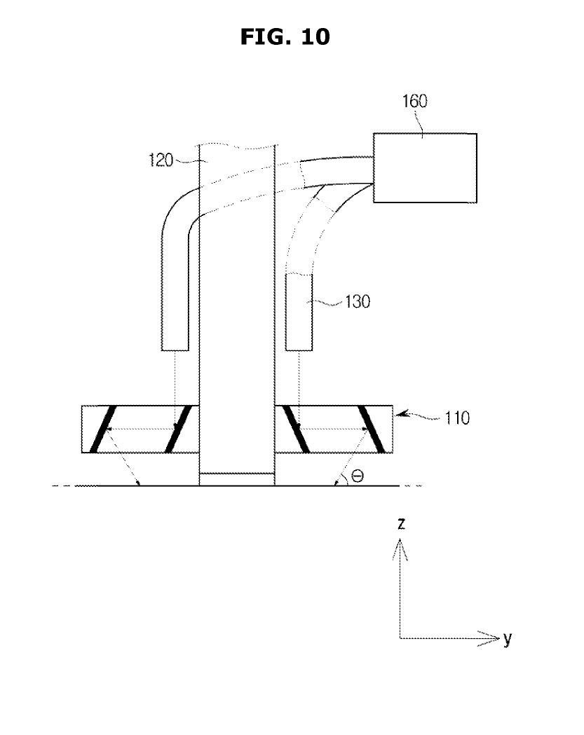

FIGS. 9 and 10 are views showing another embodiment of the optical system applied to the photoacoustic probe module of the above described embodiment.

As exemplarily shown in FIG. 9, to guide the laser beam transmitted by the optical fiber 130, the photoacoustic probe module 100 may include a prism as the optical system 110. The prism has a feature of reflecting incident light, thus serving to guide a laser beam introduced thereinto.

Referring to FIG. 10, after a target incidence angle is determined, a prism corresponding to the target incidence angle may be used as the optical system 110. Specifically, a prism, which may reflect an incident laser beam such that the laser beam is introduced into the object at a target incidence angle, may be used. Alternatively, a prism, which has a different reflection angle depending on a laser incidence position, may be provided. In this case, the prism may be rotated to achieve a target laser incidence angle.

FIGS. 11A, 11B, and 11C are views explaining control of an internal depth of the object, at which the laser beam arrives, which may also be called a depth point, by the optical system in accordance with another embodiment.

To vary an internal depth of the object, at which the laser beam arrives, a position of the prism may be shifted. Specifically, similar to the optical system 110 slidably coupled to move in a longitudinal direction along the photoacoustic probe 120, the prism may also move in a longitudinal direction along the photoacoustic probe 120 to control the internal depth of the object at which the laser beam arrives. FIGS. 11A to 11C illustrate variation in the depth at which the laser arrives via movement of the prism along the photoacoustic probe 120.

The prism is moved in the Z-axis to be closer to the surface of the object from a position as shown in FIG. 11A to a position as shown in FIG. 11C. As a result, it will be appreciated that the internal depth of the object at which the laser beam arrives increases in the order of d₁, d₂, d₃.

To set the optical system 110 to guide the laser beam to a target depth, the user may directly move the prism, or the prism may be moved by internal operation of an apparatus.

As mentioned above, instead of the prism, the optical system 110 of FIG. 5 may be moved. Guiding the laser beam to a target depth by moving the optical system 110 including the first mirror 112 and the second mirror 113 is equal to that in the prism.

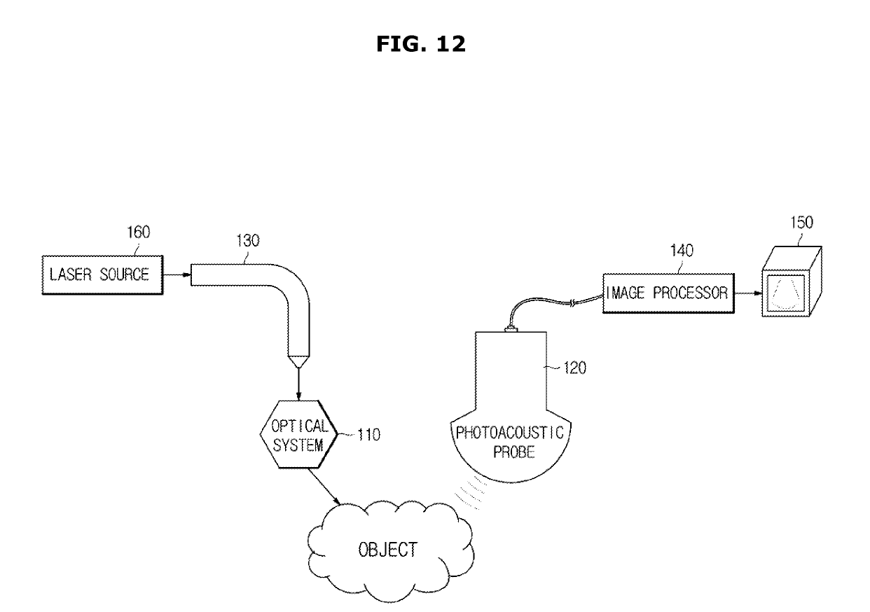

FIG. 12 is a block diagram showing a photoacoustic imaging apparatus including the photoacoustic probe module in accordance with an exemplary embodiment.

Referring to FIG. 12, the photoacoustic imaging apparatus in accordance with one embodiment may include a laser source 160 to generate a laser beam, the photoacoustic probe module including the optical system 110 to guide the laser beam such that the laser beam arrives at a target internal depth of the object at a target incidence angle and the photoacoustic probe 120 to receive acoustic waves emitted from the target depth at which the laser beam has arrived, and an image processor 140 to produce a photoacoustic image based on the received acoustic waves. The photoacoustic probe module may further include the optical fiber 130 to transmit the laser generated by the laser source 160 to the optical system 110. In addition, the photoacoustic imaging apparatus may include a display 150 to display the photoacoustic image produced by the image processor 140 on a screen.

The laser source 160 may generate a laser beam for production of a photoacoustic image. The laser source 160 may be a semiconductor laser diode (LD), light emitting diode (LED), or solid laser or gas laser emitting device, which may generate a laser beam having a specific wavelength component or monochromatic light containing the specific wavelength component. Alternatively, the laser source 160 may include a plurality of laser sources to generate coherent electromagnetic waves having different wavelengths.

In one example, when the photoacoustic imaging apparatus is used to measure the hemoglobin concentration of an object, a laser beam having a pulse width of approximately 10 ns may be generated using a Nd—YAG laser (solid laser) having a wavelength of approximately 1,000 nm or a He—Ne gas laser having a wavelength of 633 nm. Although hemoglobin concentration in the body varies optical absorption according to the type of hemoglobin, generally, laser light within a wavelength of 600 nm to 1,000 nm may be absorbed. Small light emitting devices, for example a laser, an LDs, or LEDs, used to generate coherent electromagnetic waves may be formed of InGaAIP with regard to a wavelength of approximately 550˜650 nm, GaAlAs with regard to a wavelength of approximately 650˜900 nm, or InGaAs or InGaAsP with regard to a wavelength of approximately 900˜2,300 nm. In addition, Optical Parametric Oscillator (OPO) lasers, which may vary a wavelength using nonlinear photonic crystals, may be used.

The photoacoustic probe module 100 may guide the laser beam generated by the laser source 160 and receive acoustic waves generated by the laser beam.

More specifically, the optical fiber 130 may transmit the laser beam generated by the laser source 160 to the optical system 110. The optical system 110 may guide the transmitted laser beam such that the laser beam arrives at a target internal depth of the object at a target incidence angle. The photoacoustic probe 120 may receive acoustic waves generated from the target depth by the laser beam.

The image processor 140 may produce a photoacoustic image based on the acoustic waves received by the photoacoustic probe module 100. Production of a photoacoustic image based on acoustic waves is known and thus a detailed description thereof will be omitted below.

The image processor 140 may be a hardware processor, such as a Central Processing Unit (CPU) or Graphics Processing Unit (GPU).

The display 150 may display the photoacoustic image produced by the image processor 140 on a screen. The user may check the internal state of the object at the target depth based on the photoacoustic image displayed on the screen, and may take an appropriate measure when abnormality is sensed.

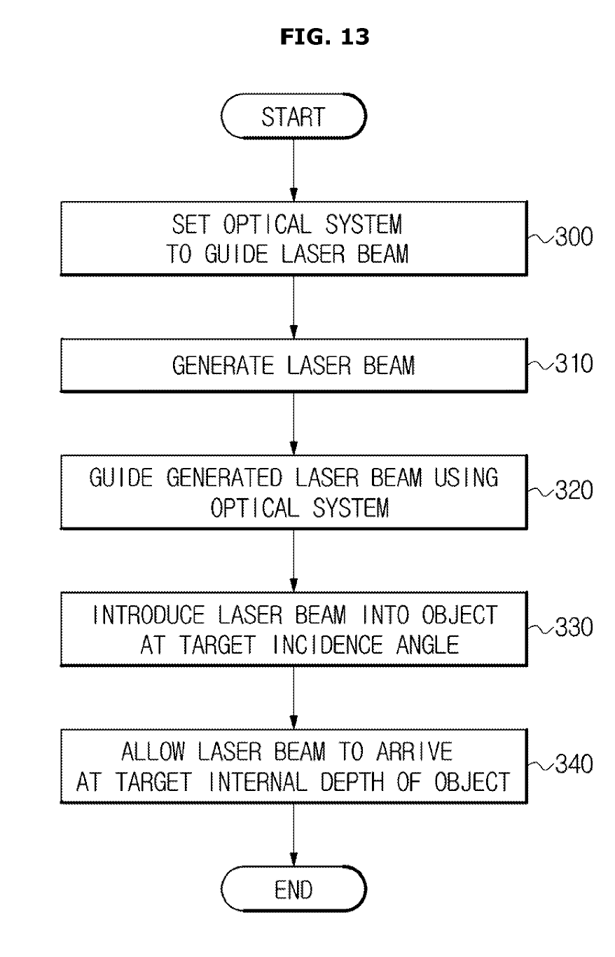

FIG. 13 is a flowchart showing a method of guiding a laser beam toward an object in accordance with an exemplary embodiment.

First, the optical system 110 may be set to guide a laser beam (operation 300). The optical system 110 may control a target laser incidence angle relative to an object and a target internal depth of the object at which the laser beam arrives. Thus, the optical system 110 may be set based on the determined target incidence angle and target internal depth of the object.

The optical system 110 may be set by the user, or may be set based on internal operation of an apparatus.

After setting of the optical system 110 is completed, the laser source may generate a laser beam (operation 310). Although the laser beam to be emitted toward the object may be a general pulsed laser beam, a continuous wave laser bmea may be emitted.

When the generated laser beam is transmitted toward the optical system 110, the laser may be guided to arrive at the target internal depth of the object at the target incidence angle (operation 320). The optical system 110 may include the first mirror 112 and the second mirror 113, or may take the form of a prism, and serves to guide the laser beam according to specific methods of the respective embodiments.

The guided laser beam is introduced into the surface of the object at the target incidence angle (operation 330), and advances in the object to arrive at the target internal depth of the object (operation 340).

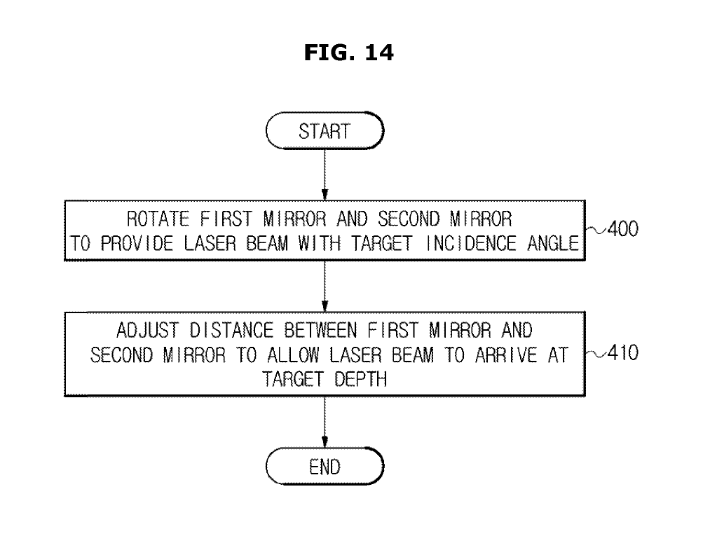

FIG. 14 is a flowchart showing a method of setting the optical system in accordance with an exemplary embodiment. The following description with reference to FIG. 14 assumes that the optical system 110 includes the first mirror 112 and the second mirror 113.

The first mirror 112 or the second mirror 113 may be rotated to provide a laser beam generated by the laser source with a target incidence angle (operation 400). As described above, because the laser incidence angle varies a laser absorption rate at the surface of the object, the first mirror 112 or the second mirror 113 may be rotated to provide the laser beam with an optimum target incidence angle.

Next, a distance between the first mirror 112 and the second mirror 113 may be adjusted to allow the laser beam to arrive at a target internal depth of the body (operation 410). An internal depth of the object, selected to produce a photoacoustic image, is set to a target depth, and a distance between the first mirror 112 and the second mirror 113 may be adjusted as well as the longitudinal distance of both mirrors relative to the object surface based on the target depth.

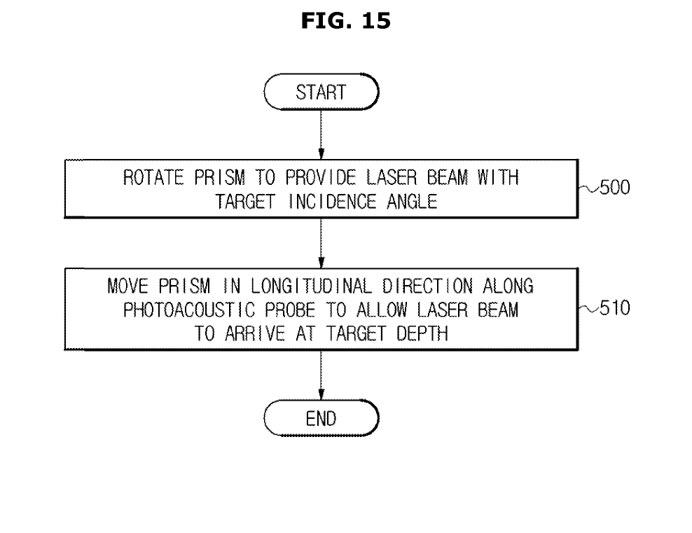

FIG. 15 is a flowchart showing a method of setting the optical system in accordance with another exemplary embodiment. The following description with reference to FIG. 15 assumes that the optical system 110 takes the form of a prism.

The prism may be rotated to provide a laser beam generated by the laser source with a target incidence angle relative to the object (operation 500). The used prism has a variable reflection angle depending on a laser beam incidence point thereof. Thus, when the prism is rotated, a laser beam incidence position of the prism varies, which causes variation in the laser incidence angle relative to the object.

Next, the prism may be moved in a longitudinal direction along the photoacoustic probe to allow the laser beam to arrive at a target depth in the body (operation 510). In this case, the prism may be longitudinally movably coupled to the photoacoustic probe. Because an internal depth of the object, at which the laser beam arrives, is variable via movement of the prism, the laser beam may be generated after the prism is fixed at a position to guide the laser beam to a target depth.

As is apparent from the above description, according to one aspect of a photoacoustic probe module and a photoacoustic imaging apparatus having the same, a laser beam to be emitted to an object is guided to a selected internal depth of the object at an optimum incidence angle, which may ensure production of a more vivid photoacoustic image of the interior of the object.

According to another aspect of a photoacoustic probe module and a photoacoustic imaging apparatus having the same, an optical system may be coupled to a conventional ultrasonic probe to emit a laser beam and receive acoustic waves without requiring a separate device. Further, the photoacoustic imaging apparatus may be used to produce an ultrasonic image or a photoacoustic image.

While exemplary embodiments have been described with respect to a limited number of embodiments, those skilled in the art, having the benefit of this disclosure, will appreciate that other embodiments can be devised which do not depart from the scope as disclosed herein. Accordingly, the scope should be limited only by the attached claims.

What is claimed is:

1. A photoacoustic probe module comprising:
an optical system configured to guide a laser beam generated by a laser source such that the laser beam arrives at an object at a target incidence angle and penetrates the object to a target internal depth; and
a photoacoustic probe configured to receive acoustic waves emitted from the target depth by the laser beam.

2. The module according to claim 1, wherein the optical system comprises:
a first mirror located along a transmission path of a laser emission direction and is configured to change a direction of the laser beam; and
a second mirror configured to reflect the laser beam, having the changed direction, toward the object such that the laser beam arrives at the object at the target incidence angle and penetrates the object to the target internal depth.

3. The module according to claim 2, wherein the first mirror and the second mirror are configured to be rotatable.

4. The module according to claim 3, wherein the optical system is further configured to guide the laser beam to provide the laser beam with the target incidence angle via rotation of the first mirror and the second mirror.

5. The module according to claim 2, wherein a distance between the first mirror and the second mirror is configured to be adjustable.

6. The module according to claim 5, wherein the optical system is further configured to guide the laser beam such that the laser beam arrives at the target depth via adjustment of the distance between the first mirror and the second mirror.

7. The module according to claim 1, wherein the optical system comprises:
a prism configured to guide the laser beam such that the emitted laser beam has the target incidence angle.

8. The module according to claim 1, wherein the optical system is coupled to the photoacoustic probe and is configured to move in a longitudinal direction along the photoacoustic probe.

9. The module according to claim 8, wherein the optical system is further configured to move along the photoacoustic probe and guide the laser beam such that the laser beam penetrates to the target depth.

10. The module according to claim 1, further comprising:
an optical fiber configured to transmit the laser beam generated by the laser source to the optical system.

11. A photoacoustic imaging apparatus comprising:
a laser source configured to generate a laser beam;
a photoacoustic probe module comprising:
an optical system configured to guide the laser beam such that the laser beam arrives at an object at a target incidence angle and penetrates the object to a target internal depth, and
a photoacoustic probe configured to receive acoustic waves emitted from the target depth by the laser beam; and
an image processor configured to produce a photoacoustic image based on the received acoustic waves.

12. The apparatus according to claim 11, wherein the optical system comprises:
a first mirror located along a transmission path of a laser emission direction and is configured to change a direction of the laser beam; and
a second mirror configured to reflect the laser beam, having the changed direction, toward the object such that the laser beam arrives at the object at the target incidence angle and penetrates the object to the target internal depth.

13. The apparatus according to claim 12, wherein the first mirror and the second mirror are configured to be rotatable.

14. The apparatus according to claim 13, wherein the optical system is further configured to guide the laser beam to provide the laser beam with the target incidence angle via rotation of the first mirror and the second mirror.

15. The apparatus according to claim 12, wherein a distance between the first mirror and the second mirror is configured to be adjustable.

16. The apparatus according to claim 15, wherein the optical system is further configured to guide the laser beam such that the laser beam arrives at the target depth via adjustment of the distance between the first mirror and the second mirror.

17. The apparatus according to claim 11, wherein the optical system comprises:
a prism configured to guide the laser beam such that the emitted laser beam has the target incidence angle.

18. The apparatus according to claim 11, wherein the optical system is coupled to the photoacoustic probe and is configured to move in a longitudinal direction along the photoacoustic probe.

19. The apparatus according to claim 18, wherein the optical system is further configured to move along the photoacoustic probe and guide the laser beam such that the laser beam penetrates to the target depth.

20. The apparatus according to claim 11, wherein the photoacoustic probe module further comprises:
an optical fiber configured to transmit the laser beam generated by the laser source to the optical system.
